(19) United States
(12) Patent Application Publication
(10) Pub. No.: US 2018/0263521 A1
(43) Pub. Date: Sep. 20, 2018

(54) SYSTEM AND METHOD FOR EMG SIGNAL ACQUISITION

(71) Applicant: TRIBE PRIVATE COMPANY, Komotini (GR)

(72) Inventor: Dimitrios STORDOPOULOS, Serres (GR)

(73) Assignee: TRIBE PRIVATE COMPANY, Komotini (GR)

(21) Appl. No.: 15/462,022

(22) Filed: Mar. 17, 2017

(51) Int. Cl. A61B 5/04; A61B 5/0492; A61B 5/00

(52) U.S. Cl. CPC A61B 5/04017; A61B 5/6802; A61B 5/7225; A61B 5/0492

(57) ABSTRACT

An analog front-end system and method for Electromyography (EMG) signal acquisition in wearable applications. The system includes filters for restricting the bandwidth of captured EMG signals, a multiplexer, an amplifier, and a microcontroller unit having an analog-to-digital converter for converting the analog EMG signals into digital signals. In the system and method, the bandwidth of the EMG signals is filtered before the EMG signals are multiplexed by the multiplexer, and the EMG signals are multiplexed by the multiplexer so that one amplifier is used for amplifying the EMG signals before they reach the analog-to-digital converter. The EMG signals are acquired by multilayered sensors applied to wearable apparel, and a connector transfers the body signals from the multilayered sensors to a central device having the analog front-end system for computing, monitoring, and sending the body signals to a display.

### FIELD OF INVENTION

The present invention relates to a new system and method for Electromyography (EMG) signal acquisition in wearable applications. The new system and method significantly outperform two previously known approaches for EMG signal acquisition in areas crucial to wearable applications, such as quality and amplitude of the signal, size, power consumption, and price.

### BACKGROUND OF INVENTION AND DESCRIPTION OF PRIOR ART

The Electromyogram (EMG) is a biosignal that is generated when a motor neuron action potential from the spinal cord arrives at a motor end plate. An sEMG signal is the summation of action potentials from the muscle fibers under the electrodes placed on the skin. Any sEMG signal can be recorded as it propagates along the muscle fibers within a muscle, through the method known as Electromyography using appropriate biosignal acquisition equipment.

One of the most important parts of a biosignal device is the analog hardware which is used to acquire, filter, and amplify the biosignal to an appropriate level.

When the biosignals are acquired from the human body by electrodes, the biosignals are very weak (small amplitudes). Because of their small amplitude, these signals have little use to any system. However, if these signals are amplified to an appropriate level they can be detected and read accordingly for analysis. The amount of amplification, referred to as the gain, is determined by the system specification and is dependent on the signal being measured as well as other circuitry requirements.

Another critical aspect of acquiring EMG signals from electrodes is the amount of noise in the signals. For proper EMG signal analysis, the noise needs to be removed from the EMG signal so that the signal can be analyzed.

Due to the aspects mentioned above, the Analog Front-End (hereafter AFE) configuration is important for the acquisition of EMG signals. An AFE configuration is considered as a set of analog signal conditioning circuitry that uses:
(a) Filters for filtering the bandwidth of the EMG signals and removing noise from the EMG signals;
(b) A combination of amplifiers for amplifying the EMG signals;
(c) Application-specific integrated circuits for sensors and other circuits to provide a configurable and flexible electronics functional block for interfacing a variety of sensors to an Analog to Digital Converter (hereafter “ADC”).

Analog front-end (AFE) configurations are used in digital systems, mostly alongside microprocessors, to interface with sensors of many kinds. Most sensor signals have inherent limitations such as weak signal output, noisy environment, quality of sensor, etc. In order to overcome the aforementioned limitations, signal conditioning is required and is performed by the analog front-end (AFE) configuration. Thus, an analog front-end (AFE) configuration is important for acquiring a signal that can be retrieved by an analog to digital converter (ADC) with small quantization error. There are generally two known analog front-end (AFE) configurations used for EMG signal acquisition.

In the first known configuration, hereafter Config1, a separate signal conditioning circuit is used per EMG signal, as depicted in FIG. 1. Each conditioning circuit includes an instrumentation amplifier (in-amp) as well as appropriate filters. A High Pass Filter (hereafter “HPF”) is used to remove DC component and low frequencies. A Low Pass Filter (hereafter “LPF”) is used mainly as an anti-aliasing filter (AAF) for satisfying the Nyquist criterion. Often, an additional amplification block is added before the Analog to Digital Converter stage. The HPF and LPF filters are active filters. The Config1 can be implemented using low power instrumentation-amplifiers (in-amps) such as INA333, AD8227, INA827, etc. In order to reduce size, this approach can use special integrated circuits (ICs) for EMG conditioning like the AD8232 in which apart from the in-amps, there are integrated operational amplifiers (op-amps) for the filtering stages.

In a second known configuration, hereafter Config2, an integrated circuit (SoC) is employed which includes eight internal ADCs and amplification circuits to capture up to eight EMG signals, as depicted in FIG. 2. In Config2, eight RC anti-aliasing filters and eight RC High Pass Filters are connected to remove unwanted frequencies. After the conversions inside the SoC, the results are transmitted through a serial interface to an external microcontroller unit (MCU) for further processing. The Config2 can be implemented using SoCs such as AD1298, AD1198, ADS131E08, etc.

WO2005/094674 discusses a biopotential measurement system which incorporates eight active electrodes. Each electrode incorporates an instrumentation amplifier (in-amp), an active low-pass filter, an active high pass filter, a variable gain amplifier, and an analog-to-digital converter. Thus, the number of instrumentation amplifiers (in-amps) and analog-to-digital converters is equal to the number of measured bio-signals. Also, WO2005/094674 uses a multiplexer after the digitation of each signal.

EP1815784 similarly discloses a system having an instrumentation amplifier, a filter, and an analog-to-digital converter connected to each bio-signal.

WO2015200802 discloses a system having one or more sensors 212, 214 that share the same analog circuitry. Inputs 222 and 224 provide analog inputs to respective multiplexers 232, 234, and multiplexers 232, 234 select specific analog inputs for amplification by amplifiers 242, 244. Amplifiers 242 and 244 provide their outputs to one or more analog to digital converters (ADCs) 256. By using the same filter 254 for all input signals, the switching time between the signals in WO2015200802 is far too long for a proper bio-signal acquisition.

EP2034887 similarly discloses an anti-aliasing filter 170 used after multiplexing and amplification. Thus, the multiplexed signals use the same anti-aliasing filter 170 resulting in an increased switch time between the signals. For the sampling rate in EP2034887, the switching time is at its limits meaning that for each cycle time, only eight signals can be at the input. The settling time of EP2034887 is approximately 200 us.

WO2010103542 discloses a multiplexer for applying self-calibration of an ECG device. The multiplexer's output is connected to a Signal Conditioning and Amplifier Unit. This unit consists of an instrumentation amplifier, a band-limit filter, and a post amplification and level-shifting circuit. WO2010103542 places the filters after the multiplexer, and thus fast switching of input signals is not possible in WO2010103542.

US2007/0010721 discloses an active sensor unit 300 in a monopolar configuration for detecting body temperature, body weight, body mass index, and body fat. The sensor unit 300 comprises sensor circuitry which includes a process sensor 311, transducer 312, signal filter 313, signal multiplexer 314, process signal reference 315, signal amplifier 316, and analog-to-digital converter 317. The signals detected in this publication are of higher amplitude, less noise, and have a simpler pattern than an EMG signal. An EMG signal is the summation of all the signals transmitted by the total number of muscle fibers within the electrodes detection range. Although each fiber's signal is the output of an electrochemical process and has a simple pattern, the total EMG signal is a complex, non-periodical, and chaotic. Therefore, acquisition of the EMG signal requires specific acquisition and treatment protocols that differ from those that are used to acquire simple body signals such as body temperature, body weight, body mass index, and body fat.

The contents of the above patents and patent application publications are hereby incorporated herein by reference.

With regard to the means for acquiring the EMG signals, EMG surface electrodes must provide high signal resolution and full-bandwidth signal detection with low noise. Noise which affects the quality of the EMG signal may result from sources such as electrical noise generated in the electronic equipment used for the detection and recording of the EMG signal, ambient noise from electromagnetic devices such as TVs or radio signals, electrical-power lines, fluorescent lamps, and the inherent instability of the EMG signal because of its quasi-random nature. Other sources of noise include movements of the electrode's detection surface in relation to the skin which changes the characteristics of the skin-electrode interface, and movements of the cables that connect the electrodes to the amplifier. Also, for wearable applications, electrodes must be able to constantly bend, flex, twist, or even stretch due to the movements of the user when exercising. It is a challenge to design electrodes that withstand such forces because cyclic stretching and relaxation can result in material fatigue.

Conventional EMG electrodes used in medical or research applications are too rigid and have too little flexibility to be comfortably worn during exercise. The stiffness of conventional electrodes makes it difficult to create good conductive interface between the electrode and the human skin because the skin has a soft, rounded, and granular surface which prevents the stiff electrode from making good conduct with the skin.

Conductive threads created from metal monofilaments incorporated into base yarns like cotton, polyester, polyamides, and aramids have been used as EMG electrodes for wearable applications. The metal monofilaments can be made out of copper, brass, bronze, silver, gold, aluminum, or other conductive materials. A typical conductive yarn has a base group of fibers (yarn) and a metal monofilament twisted around them.

Another method for creating wearable EMG electrodes has been to coat conductive elements such as, for example, nanoparticles of copper, silver, gold, stainless steel onto a base of yarn made from cotton, polyester, polyamides or aramids. These coatings can be applied to the surface of fibers, yarns, or even fabrics to create electrically conductive textiles. Common textile coating processes include spin coating, electro-less plating, evaporative deposition, and sputtering for coating the textile with a conductive polymer. The aforementioned techniques for creating wearable electrodes for apparel have several significant drawbacks. For example, electrodes made from conductive threads or coatings of conductive elements cannot withstand repeated strenuous conditions such as machine washing and tumble-drying, or the constant stretching/twisting when being worn by user.

Thus, electrodes made from conductive threads or coatings tend to wash away, oxidize, and/or break-off from the wear and tear of the fabric fibers. This causes the electrodes to lose their conductivity. Materials that do not oxidize such as, for example gold, would make the electrode too expensive to manufacture for commercial use on a large scale. Further disadvantages include that the electrically conductive fabric tends to be hard and rigid such that it is not comfortable to wear. This also creates a poor conductive interface due to the random and uneven surface and rigidness of the human-electrode interfaces.

To permanently fuse the electrodes and wiring made from conductive threads, it is necessary to sow them to the apparel. This requires additional stitching, and hence reduces the stretchability of the apparel and the aesthetics of the apparel. Also, this will cause the apparel to be heavy, stiff, and inflexible due to the bulkiness and stiffness of the conductive threads and coatings. Thus, because neither conductive threads nor coatings have the same flexibility as the fabrics and textiles typically used for sports apparel, these types of electrodes hinder motion, functionality, and usability of the article of apparel.

Other drawbacks of integrating conductive yarns into wearable piece of fabric is that the process is complex and seldom uniform which makes the process expensive and difficult to replicate at a consistent level of quality. Also, it is difficult and expensive to insulate conductive-multi-filaments/fibers and electrodes made from conductive threads and coatings require a special manufacturing processing with specialized equipment and personnel. Thus, integrating conductive threads and coatings into athletic apparel leads is expensive due to additional manufacturing and handling costs.

### SUMMARY OF THE INVENTION

In accordance with a first embodiment of the invention, there is provided an analog front-end system for the acquisition of EMG signals for assessing the health of muscles and nerve cells that control the muscles, comprising means for acquiring the plurality of EMG signals and delivering a differential signal for each of the plurality of EMG signals; a first set of filters comprising a first filter for each of the plurality of EMG signals; a multiplexer; an amplifier; and a microcontroller unit comprising an analog-to-digital converter. In this embodiment, the bandwidth of each of the plurality of EMG signals is filtered by the first set of filters before the EMG signals are multiplexed by the multiplexer, and the EMG signals are multiplexed by the multiplexer so that a single amplifier is used for amplifying the EMG signals before they reach the analog-to-digital converter to provide a gain of at least 250 times, and a quantization error of less than 0.012%.

Means for acquiring the plurality of EMG signals and delivering a differential signal for each of the plurality of EMG signals include body sensors and/or electrodes that can measure EMG signals from all over the body of a user. The body sensors and/or electrodes are stretchable and of low resistance so that they can be implemented into sports apparel, without compromising on the accuracy of the measurements taken by the system. Contact by the body sensors and/or electrodes with the skin of a user can be achieved by elastic stress and strain.

In one embodiment, the means for acquiring the plurality of EMG signals and delivering the differential signal for each of the plurality of EMG signals include multilayered signals having a first protective insulation layer of non-conductive insulator ink; a protective conductive layer and a conductive layer forming a pair of electrodes; a second protective insulation layer; and an adhesion layer. The conductive layer of the multilayered signals is made from a highly conductive-ink for capturing biometric signals. The protective insulation layer of the multilayered signals is printed onto a heat-transfer film for use as a transport agent for the multilayered signals, and the multilayered signals permanently bond to a fabric when the heat-transfer film to which the multilayered signals are printed onto is heat-pressed onto the fabric.

The present invention utilizes a multiplexer for acquiring the multiple EMG signals so that a single amplifier is used. The implementation of the multiplexer into the architecture of the present invention presented many challenges and obstacles. For example, the resistance from a multiplexer can disturb the measurement of an EMG signal, and other problems include increased switch time between the EMG signals and increased power consumption.

These problems are overcome in the present invention by using a multiplexer with low resistance and an amplifier with high slew rate and fast settling time so that switching between the signals is fast thereby lowering power consumption. Also, the EMG signals pass through different filters even though the signals pass through the same amplifier. Filters have time constants, and the time constant of a filter depends on the frequency band that it filters. In the case of the present invention (EMG signal filtering), this is some milliseconds. Thus, if the same filter would be used for all the EMG signals, the switch time between the EMG signals would be some milliseconds. Instead, the switch time between the EMG signals should be less (some microseconds).

The analog front-end system can further comprise a second set of filters comprising a second filter for each of the plurality of EMG signals. Thus, in this embodiment, each EMG signal passes through a first filter from the first set of filters and through a second filter from the second set of filters to restrict its bandwidth before being multiplexed by the multiplexer. The first set of filters can be passive anti-aliasing filters and the second set of filters can be passive high pass filters. In one embodiment, the first and second sets of filters restrict the bandwidth of each EMG signal to a frequency range of 20 Hz to 100 Hz. After being filtered by the first and second filters, the EMG signals are then multiplexed by the multiplexer which may be an analog switch. The operation of the multiplexer may be controlled by the microcontroller unit (MCU).

In one embodiment of the present invention, the first set of filters comprises eight first filters and the second set of filters comprises eight second filters for processing eight EMG signals. In other embodiments, the number of first and second filters may vary according to the number of EMG signals being processed. Therefore, there could be fewer than eight first filters or more than eight first filters. Similarly, there could be fewer than eight second filters or more than eight second filters. However, notwithstanding the number of EMG signals being processed, the present invention comprises a single amplifier. In one embodiment, the amplifier is a low power instrumentation-amplifier with high Common Mode Rejection Ratio (CMRR).

In one embodiment of the present invention, the multiplexer, the single amplifier, and the microcontroller unit are part of an integrated circuit. The multiplexer and amplifier are differential and a transducer, a process signal reference, or a biocatalyst signal reference are not used for the EMG signal acquisition.

In some embodiments, the analog front-end system is implemented into a central device that can be attached to sports apparel of any kind by a connector for long term monitoring of several bio-signals during all kinds of physical activity.

In accordance with yet another embodiment of the present invention, there is provided a method for processing a plurality of EMG signals for assessing the health of muscles and nerve cells that control the muscles comprising the steps of: acquiring a differential signal for each of the plurality of EMG signals, applying a first set of filters comprising a first filter for each of the plurality of EMG signals to restrict the bandwidth of each of the plurality of EMG signals to a predetermined frequency range, multiplexing the filtered EMG signals by an analog multiplexer switch, amplifying the multiplexed EMG signals by a single differential amplifier, and sending the amplified EMG signals to a microcontroller unit for processing. The bandwidth of each of the plurality of EMG signals is filtered by the first set of filters before the EMG signals are multiplexed by the multiplexer, so that a gain of at least 250 times and a quantization error of less than 0.012% is obtained before the EMG signals are processed by the analog-to-digital converter.

In one embodiment, a second set of filters comprising a second filter for each of the plurality of EMG signals is applied before multiplexing the signals. In this embodiment, the first and second sets of filters restrict the bandwidth of each of the plurality of EMG signals to a frequency range of 20 Hz to 100 Hz. In some embodiments, the first filter is a passive anti-aliasing filter and the second filter is a passive high pass filter.

In one embodiment, eight EMG signals are acquired, filtered, and multiplexed by the method of the present invention. In other embodiments, fewer than eight EMG signals or more than eight EMG signals are acquired, filtered, and multiplexed. However, notwithstanding the number of EMG signals being processed, a single amplifier is used to amplify the EMG signals. In one embodiment, the amplifier is a low power instrumentation-amplifier with high Common Mode Rejection Ratio (CMRR).

### BRIEF DESCRIPTION OF THE DRAWINGS

FIG. 1 shows a first known configuration (Config1) for an acquisition process which includes one signal conditioning circuit per EMG signal, and one ADC.

FIG. 2 shows a second known configuration (Config2) for an acquisition process for eight EMG signals which includes eight RC anti-aliasing filters, eight RC High Pass Filters, and one SoC integrated circuit which consists of eight amplifiers and eight ADCs.

FIG. 3 shows the configuration of the present invention (Config3) for acquisition of signals from eight EMGs which includes eight RC anti-aliasing filters, one analog Multiplexer, one in-amplifier, and one ADC.

FIG. 4 shows the slew rate effect on a square wave where the broken line is the desired (ideal) output and the solid line is the actual output pulse.

FIG. 5 shows the settling time of an amplifier or other device.

FIG. 6 shows the frequency response of a single pole HPF (DC blocking) circuit.

FIG. 7 shows frequency responses of two-pole, three-pole HPF (DC blocking) circuits.

FIG. 8A shows a multilayer sensor, and FIG. 8B shows a multilayer interface for merging several sensor leads together.

FIG. 9A shows a heat-transfer film being aligned to an article of apparel, FIG. 9B shows the heat-transfer film and the apparel aligned and firmly secured together, and FIGS. 9C, 9D, and 9E show the heat-transfer film wrapped around the article of apparel, respectively.

FIGS. 10A, 10B, and 10C show the inside view of a pair of respective pairs of athletic compression shorts (apparel) with sensors embedded thereto.

FIG. 11 shows a transparent and cut-away view of a connector and a central device.

FIG. 12 shows an exploded view of the connector and central device shown in FIG. 11.

FIG. 13 shows an exploded view of the connector and central device shown in FIG. 11.

FIG. 14 shows an assembled connector with the central device inserted therein.

FIG. 15 shows an article of apparel before sensor integration.

FIG. 16 shows a heat transfer film with sensors and sensor leads printed thereon.

FIG. 17 shows heat-pressing the heat transfer film to transfer and permanently bond the sensors and sensor leads to the apparel.

FIG. 18 shows several sensor leads connected to a printed circuit board (PCB).

FIG. 19 shows an interior view of the apparel with an interior cover placed over the connection of the several sensor leads to a printed circuit board (PCB).

FIG. 20 shows another interior view of the apparel.

FIG. 21 shows an exterior view of the sealed connector on a piece of apparel.

FIG. 22 shows an exterior view of the central device being plugged into the connector.

FIG. 23 shows the central device successfully plugged-into the connector for creating a connection between the central device and the sensors.

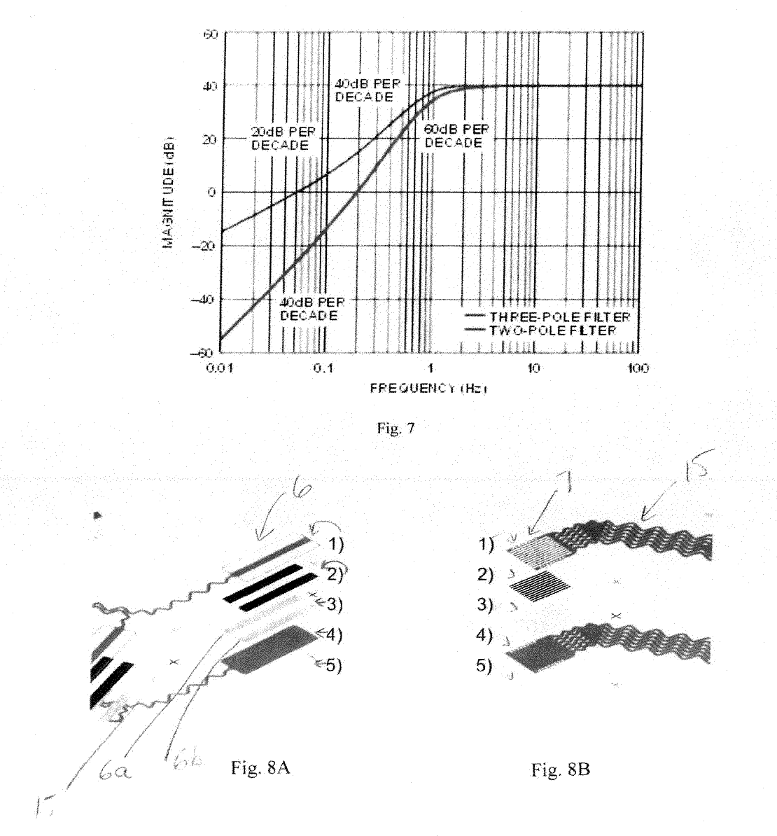

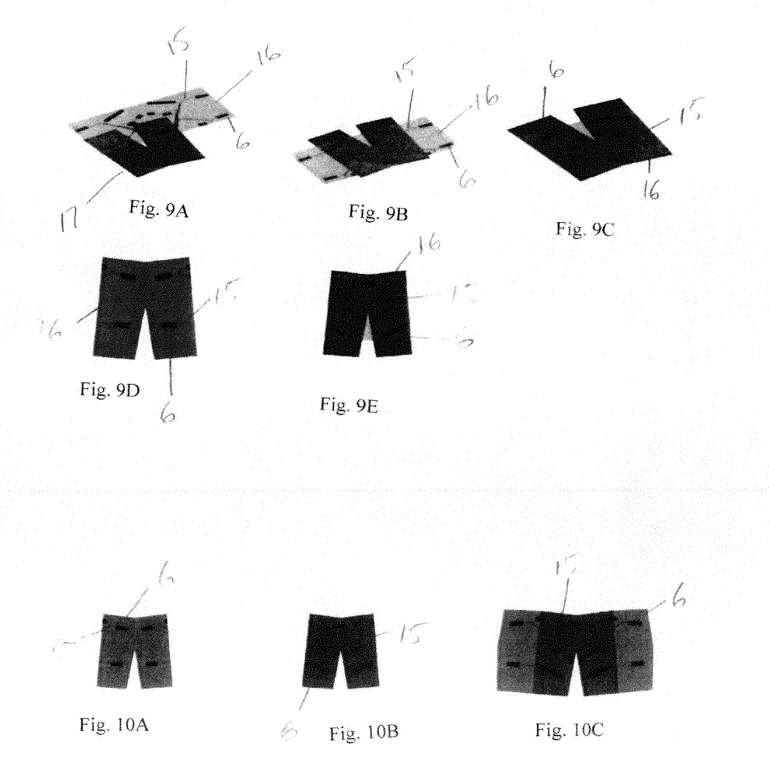

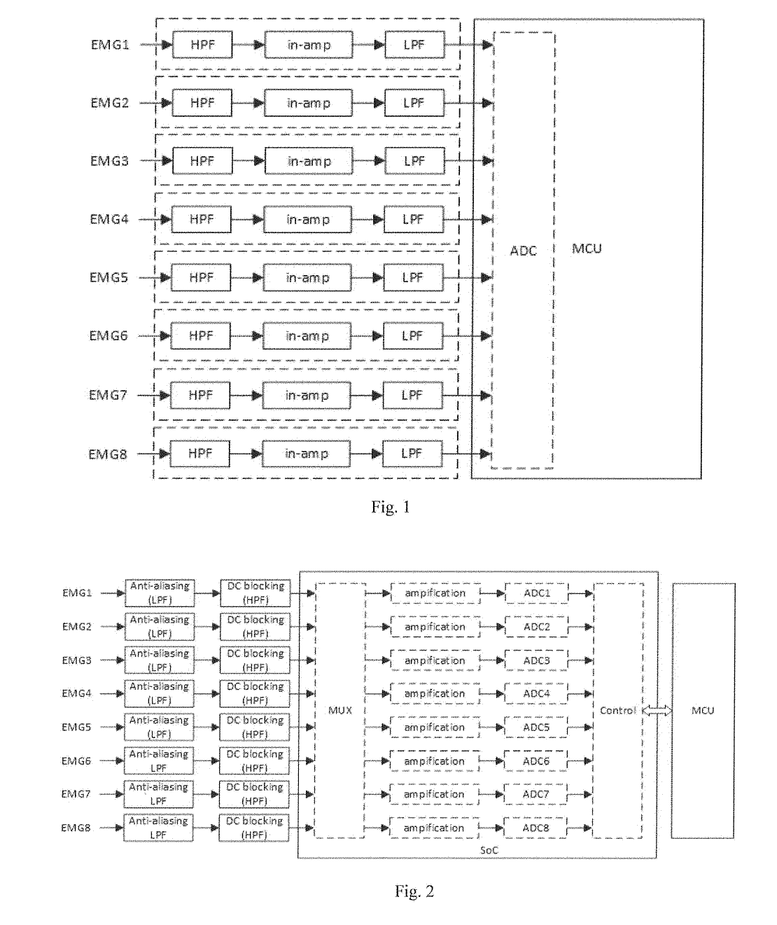

### DETAILED DESCRIPTION OF THE INVENTION

In the configuration of the present invention (Config3), filters are used to restrict the bandwidth of the EMG signals to remove high frequencies. The EMG signals are then multiplexed by an analog multiplexer (switch) before their amplification. The operation of the analog multiplexer (switch) is controlled by a microcontroller (MCU). After multiplexing, the EMG signals are then driven to an amplifier and the output of the amplifier is connected to one input of the microcontroller's analog to digital converter. In the architecture of the present invention, each EMG signal is filtered before multiplexing is done, and each EMG signal has its own filter(s). This leads to faster switching between the signals. This lowers power consumption, increases the speed of the multiplexing, and minimizes the interference between the signals. In the present invention, the settling time is as low as 15 μs. Also, the present invention is flexible because more signals and different sampling times can be added according to the equation fs<=1/(NumberOfEMGChannels*AFESettlingTime).

Because of the complex nature of the EMG signal, a differential configuration is needed in order to accurately measure the EMG signal. In a differential configuration, two signals are taken within close proximity of one another and the measured EMG signal is the subtraction of the two signals. The advantage of a differential configuration is that the common noise between the two electrodes is eliminated and, thus a cleaner EMG signal with better signal-to-noise ratio is acquired. A monopolar configuration will not give correct information about muscle activity.

In the present invention, all EMG signals share the same instrumentation amplifier (in-amp) and the same analog-to-digital converter which significantly reduces the size of EMG signal acquisition system. Thus, in the present invention, the multiplexer is used before the digitation of each signal so that only one instrumentation amplifier (in-amp) and one analog-to-digital converter are used which reduces the size and power consumption of the system.

The present invention uses a differential amplifier for EMG signal processing. This means that each input in the amplifier is an EMG signal input, because the EMG signal is a deferential signal, and a differential amplifier rejects the common noise between the two EMG signals. A simple amplifier does not reject the common noise; rather it's amplifying the noise.

The present invention uses only one amplifier. The present invention also receives a reference signal from a microprocessor without the need of an extra third electrode. Additionally, the present invention does not need a transducer. Thus, the number of components in the architecture of the present invention is significantly reduced thereby achieving a smaller size and a lower cost for an analog front-end. Further reduction in size of the analog front-end of the present invention can be achieved by using an integrated circuit (SoC) that incorporates the multiplexer, amplifier, microcontroller (MCU), and analog to digital converter. Reducing the number of components also leads to lower power consumption by the analog front-end.

EMG signal filtering by the present invention is done by a first set of filters. Optionally, a second set of filters could be used for further filtering. The EMG signals may pass through the first set of filters first, and may then pass through the second set of filters afterwards. The first set of filters may be passive anti-aliasing filters (AAF) or passive low pass filters (LPF). The second set of filters may be passive high pass filters (HPF) or passive DC blocking filters. Both the first and second filters may be used to restrict the bandwidth of the EMG signals for satisfying Nyquist criterion.

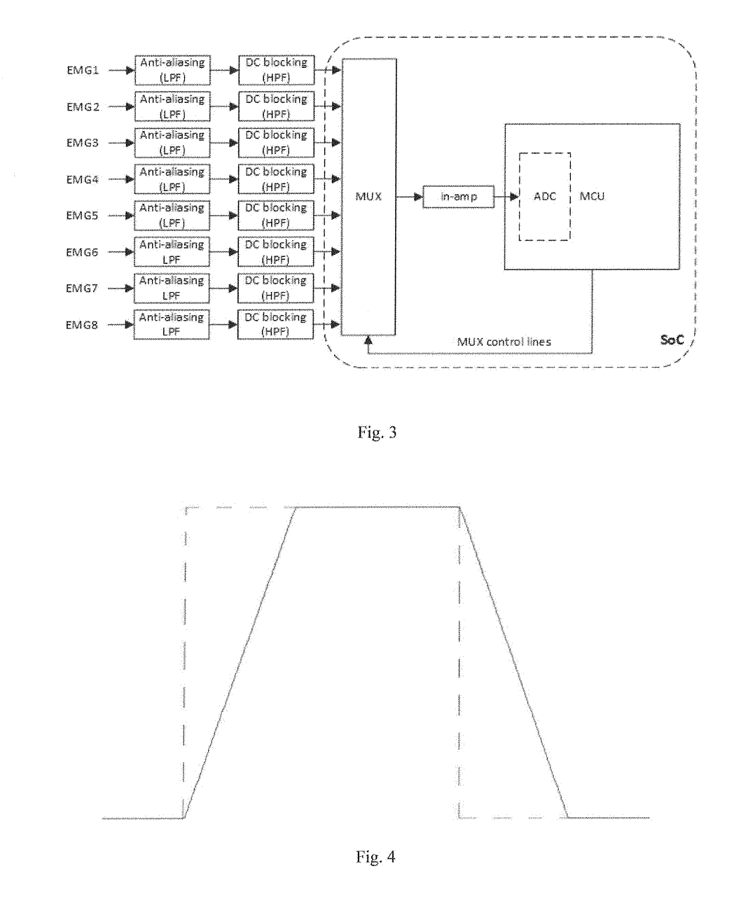

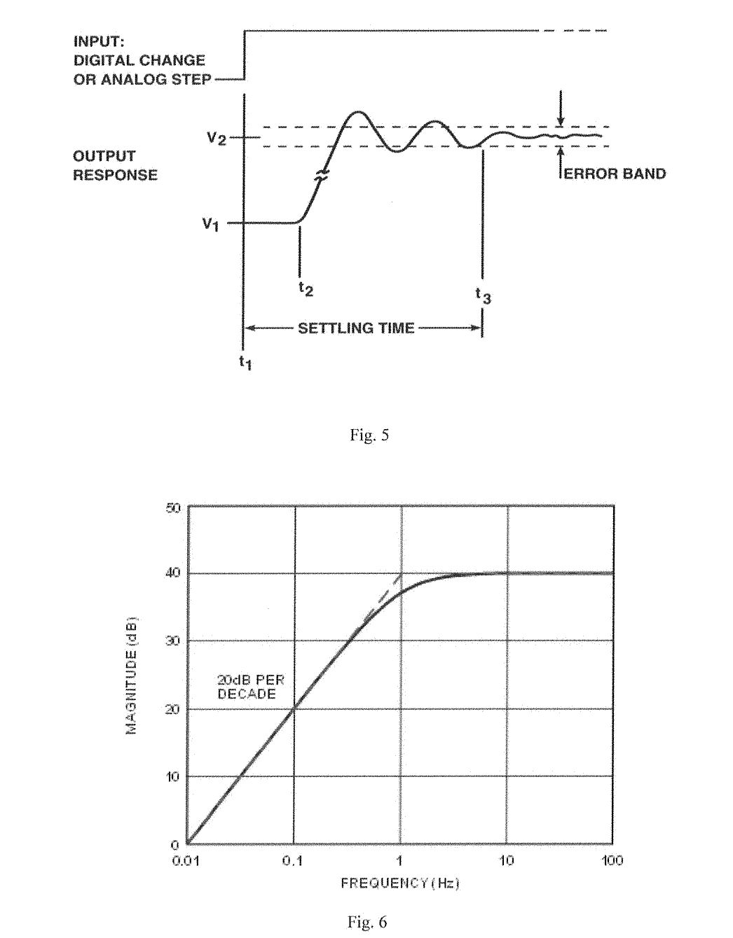

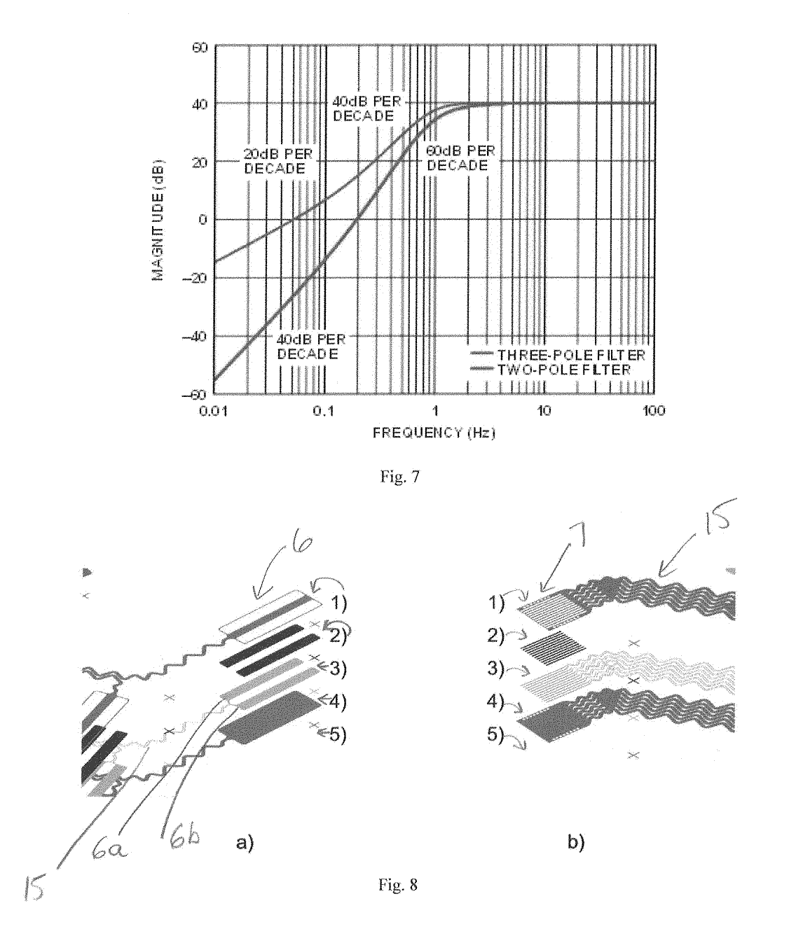

Although eight first filters and eight second filters for processing eight EMG signals are shown in FIG. 3, it is contemplated that the number of first and second filters may vary in accordance with the present invention. Therefore, there could be fewer than eight first filters or more than eight first filters. Similarly, there could be fewer than eight second filters or more than eight second filters.

The choice and frequency band of the first set of anti-aliasing filters (AAF) filters depends on the quality of the EMG electrode/sensor, the noise of the EMG electrode/sensor environment, and the quality of the EMG signal acquired by the EMG electrode/sensor itself The EMG signals acquired by the EMG electrodes/sensors can contain much unwanted information (e.g., noise).

There are also some other issues that affect the useful EMG bandwidth. For example, motion artifacts can arise due to movement of the cable that connects the EMG electrode with the conditioning circuit. Also, the detection interface between the EMG electrode and the skin could be a source of motion artifacts. These motion artifacts could cause noise at very low frequencies near the DC, such as baseline wandering, and should therefore also be removed.

The purpose of the first set of anti-aliasing filters (AAF) filters is twofold. For example, the first set of AAF filters can be used to restrict the bandwidth of an EMG signal to a specific frequency band. This way the amplification stage will amplify a signal containing the most useful information of the original EMG signal. Second, the first set of anti-aliasing filters (AAF) filters can be used to remove higher frequencies that can cause aliases in the subsequent digital processing.

The second set of HPF filters is also used to restrict the bandwidth of the EMG signal to a desirable range. The HPF filters filter out low frequencies in contrast to the anti-aliasing filters (AAF) filters which filter out high frequencies. The anti-aliasing filters (AAF) filters can be referred to as low pass filters (LPF) and the high pass filters (HPF) can be referred to as DC blocking filters.

The cutoff frequencies of the filters are important. The usable energy of an sEMG signal is limited to the 0-500 Hz frequency range. The dominant energy is located in the 20-150 Hz frequency range.

The ambient radiation is a dominant source of noise and it arises at 50 Hz or 60 Hz, depending on the regional power grid frequency. This noise should also be filtered. One of the most important filters, no matter what the frequency range of the biosignal is, is the 60 Hz (or 50 Hz outside of North America) band stop filter, also known as notch filter. This filter removes the noise that is produced from the common AC wall outlet.

Also, another important consideration is the inherent instability of the EMG signal. The amplitude of the EMG signal is quasi-random in nature and frequency components are unstable in 0-20 Hz frequency band. At this frequency range, noise from outside sources very likely exists such as slow varying drifts and potentials.

In one embodiment of the present invention, the 20 Hz to 100 Hz frequency range is selected as the desired bandwidth range for the EMG signals.

An issue encountered when processing EMG signals comes from the fact that the EMG signals are generally of very low amplitude (peak-to-peak 0-10 mV). Thus, in order to acquire and process the EMG signal reliably, the useful signal information should be amplified to an acceptable voltage level. Consequently, amplification in an analog way is done which involves the implementation of amplification circuits with high gain and high Common Mode Rejection Ratio (hereafter CMRR).

In analog to digital conversion (ADC) applications, the dynamic range is the ratio of the Root Mean Square value (hereafter “RMS”) of the full scale to the RMS noise. It indicates the range of signal amplitude that an analog to digital converter can resolve. For increased dynamic range, a low noise amplifier can be added to condition the signal to attain full scale. If the signal is too small, the useful information of the signal will be lost because of the converter's quantization noise and error.

With regard to timing, the slew rate and settling time of the amplifiers should be taken into consideration. An amplifier with fast response and the ability to propagate the EMG signals very quick in order to reduce the acquisition time as much as possible and put the circuitry into sleep is desirable. See FIG. 4.

In the present invention, a low power instrumentation amplifier (hereafter in-amp) with high Common Mode Rejection Ratio (CMRR) value and fast response is used to amplify the EMG signal and remove common mode noise. An instrumentation amplifier (in-amp) is a type of differential amplifier that has been outfitted with input buffers which eliminate the need for input impedance matching. Additional characteristics of instrumentation amplifiers (in-amp) include very low DC offset, low drift, low noise, very high open-loop gain, very high common-mode rejection ratio and very high input impedances. Instrumentation amplifiers are used where great accuracy and stability of the circuit are required. Furthermore, the EMG signal is a differential signal and the instrumentation amplifier (in-amp) will transform the EMG signal to a common ground signal. The resulting signal can then be connected to the analog to digital converter (ADC) input. The type of analog to digital converter (ADC) and the number/type of amplifiers used are the main contributors in power consumption in an AFE system.

After the amplification of the EMG signal, the resulting signal is driven to an analog to digital converter (ADC) in order to apply digital processing such as: IIR filters, mathematical calculations, wireless transmission etc. The analog to digital converter (ADC) can be a delta-sigma converter or a SAR converter. A SAR analog to digital converter has low latency time, low power consumption, and low cost, but requires input signals of high amplitude. A delta-sigma analog to digital converter can handle signals of lower amplitude, has high resolution, and can be of low power. However, disadvantages of delta-sigma converters include low speed and cycle latency.

Pipeline analog to digital converters are not considered for use in the present application because of their characteristics which in general are lower resolution, have pipeline delay/data latency, consume more power, and produce noisier results.

Another component that consumes considerable power, even if it is not technically a part of the analog front-end circuit, is the microcontroller (MCU). Thus, it is desirable for the microcontroller (MCU) to be in sleep mode for long time intervals. In order to achieve this, fast circuitry in the analog front-end (AFE) must be implemented.

The analog front-end system of the present invention can be used in a wearable application such that it can be implemented into the fabric of any kind of sports apparel. When implemented into a wearable application, the present invention can measure EMG signals from all over the body. This is feasible because the hardware of the present invention allows for a topology that distributes the sensors all over the body. The sensors used for a wearable application of the present invention are stretchable and of low resistance so that they can be implemented into sports apparel, without compromising on the accuracy of the measurements taken by the system.

The sensors are fused into the apparel by, for example, heat press, and in this way the skin-sensor contact is achieved because of the mechanical properties of the fabric (its elastic stress/strain). Moreover, because of its small size and better signal quality, the architecture of the present invention can be used in a wearable application for long term monitoring of several bio-signals during all kinds of activities and with any sensor topology or configuration.

The quality and amplitude of the EMG signal are important parameters for a wearable application. Regarding the Config2 integrated circuit (SoC) approach, the gain of the amplification circuit is up to 24 (depending on the selected SoC). This is a low gain. In the Config1 and the configuration of the present invention (Config3), the gain is much higher. For example, the gain in these configurations can be selected from a range between 100 and 1000. Therefore, with regard to the gain of the EMG signal, the Config1 and the configuration of the present invention (Config3) considerably outperform the Config2 solution.

In addition, the quality of the acquired EMG signal in Config1 and the configuration of the present invention (Config3) is also better than the quality in Config2 for at least one other reason as well: instrumentation amplifiers (in-amps) with high CMRR are used providing very good results in rejecting common mode noise.

In addition, there are two possible electrode configurations for acquiring the EMG signal which is typically acquired by placing electrodes on the muscle being analyzed. The first electrode configuration is the most common configuration and uses three electrodes. The first two electrodes are placed on a muscle and the third is placed on a neutral point on the body. The potential of the third electrode is considered of zero value (ground or reference electrode). The electronic circuit creates a signal ground reference, which constitutes a voltage level usually in the middle of the battery's voltage limits. The potential is connected to the reference electrode and the human body is driven by the device. This is normally used in order to define a common ground point between the device and of the human body skin. The electrical activity measured by the three electrodes is then sent to a n amplifier, and the amplifier eliminates random voltages caused by electrical noise by subtracting the signal of the ground electrode from the muscle electrode producing the raw EMG signal. In the second electrode configuration, only two electrodes are mandatory. The differential measurement is driven to the signal conditioning circuit. The third electrode is considered as optional. The third electrode could be used as noise cancellation or as a reference, but the third electrode is often removed because fewer electrodes mean lower cost and simpler apparel design, and noise resulting from small drifts of the ground electrode is eliminated.

Regarding Config2 for the analog front-end system, there is the option to connect a third electrode as a reference. The connections (schematic) are fixed and cannot be changed. For the Config2, the connection of the third electrode could occasionally add extra noise, which is not desired and that is why it should be used only as voltage reference and not as a noise cancellation.

In Config1 and the configuration of the present invention (Config3), there is the flexibility of using or not using the third electrode. The results are of higher quality regardless of the use of the third electrode. Therefore, Config1 and Config3 of the present invention outperform Config2 as it concerns the use of the third electrode.

With regard to filtering, the slope of a filter is defined by how many decibels the filter gain drops off per (logarithmic) frequency interval above the cutoff frequency. The interval is usually measured in decades (10) where the unit of the slope is decibels per decade (dB/dec) while the slope depends on the order of the filter. The order of the filter refers to the number of components (capacitors, inductors) that affect the “steepness” of the filter's frequency response. Higher order means higher number of components, and thus higher cost and bigger size (See FIG. 6).

Passive filters require no power supply, are not restricted by the bandwidth limitations of an amplifier, and generate little noise, merely the thermal noise from the resistive components. And with careful design, the noise's amplitude can be very low, especially when compared with circuits using active gain elements. Active filters lack inductors, thereby reducing the problems associated with those components. Active filters are also easy to design and offer the added benefit of amplifying the signal, but active filters also add noise due to the amplifying circuitry. Config1 incorporates active HPF and LPF filters while Config2 and the configuration of the present invention (Config3) use passive RC HPF and LPF filters.

The configuration of the present invention (Config3) uses no inductors and amplifies the signal in a later stage. This gets the best benefits of both filter topologies. Regardless of configuration, a digital filtering process is undertaken by the microcontroller (MCU) to reduce the size and cost that an increased number of passive components would produce, for a higher order active or passive filters design. In the case of the present invention, a 4th order BPF and 6th order notch filter for every EMG signal are implemented to derive EMG signals with the desired quality.

It should be also mentioned that active filters have the ability to amplify the signal while passive filters cause a small attenuation. In configurations where passive filters are used, the amplification of the signal is done in a later stage.

According to the measurements, the quality of the signal for Config2 and the configuration of the present invention (Config3) which use passive filters is good. Furthermore, according to the measurements of the signal quality in Config1 and the configuration of the present invention (Config3) was equally good for both implementations. By using active filters, the analog front-end (A F E) suffers from increased size and power consumption. Using passive filters instead, cost is reduced, power dissipation is improved, and no detectable differentiation in signal quality compared to the active filter configuration is exhibited. For the passive filters configuration, resistance packs can be used instead of discrete components which results roughly to a 20% size reduction. On the other hand, active filter configuration requires one op-amp per filter which results i n a higher cost, size, and consumption. Thus, the configuration of the present invention (Config3) is advantageous, as it provides a signal of equal quality, reduced size, much improved power consumption and exceptionally lower cost.

Another big advantage of the multiplexing of the present invention is that extra EMG signals can be added without the need to add more instrumentation-amplifiers and analog to digital converts. Rather, only extra switches are added. In Eq. (1), the maximum sampling frequency in respect to the number of the EMG channels and the analog front-end settling time is quoted.

fs<1/(NumberOfEMGChannels*AFEsettlingTime) (1)
fs: sampling frequency
AFEsettlingTime: in-amp propagation delay+switches propagation delay+ADC acquisition time, measured in seconds

In Table 1, the maximum sampling frequency is presented in respect to various settling times and employed EMG channels for the multiplexing solution of present invention (Config3). This table shows that the higher gain, the longer the settling time. In the configuration of the present invention (Config3), the addition of more EMG channels does not affect the sampling frequency as the number of EMG channels remain below 66 considering a gain of 100.

TABLE 1
Maximum sampling frequency in respect to the number
of EMG channels and the in-amp settling time

| fs | | Gain | Settling time | Number of EMG Channels |
|---|---|---|---|---|
| 1 | Ksps | 100 | 15 us | 66 |
| 1 | Ksps | 300 | 25 us | 40 |
| 3.333 | Ksps | 100 | 15 us | 20 |
| 3.333 | Ksps | 300 | 25 us | 12 |

The usual case for wearable devices is to be worn as accessories or be part of apparel, both of which dictate a very small form factor, making size one of the most important aspects in wearables technology.

In the Config1 architecture, 8 in-amps are used as well as 8 filtering circuits, one per EMG signal. For every additional EMG channel, an extra op-amp is needed. The big size which is required for this AFE is its main drawback.

With regard to Config2, the size of the configuration is smaller. There is one SoC that includes 8 amplification circuits and ADCs. Thus, even though Config2 comprises 8 amplification circuits having a lot of components, the size of Config2 is still smaller than that of Config1. Config2 has inputs for a maximum of eight EMG signals if one chip is employed. The number of demanded chips, n, is determined by Eq. 2 presented below.

n=ceil(EMGSignals/8) (2)
ceil: the smallest integer not less than (EMGSignals/8)

Thus, for example, when 10 EMG signals are acquired, 2 SoC ICs are required, doubling both the cost and the size for Config2.

The multiplexing solution of the present invention (Config3) has the smallest size of the three configurations. There is only one in-amp employed by the present invention (Config3) regardless of the number of the captured EMG signals. This gives a comparable advantage especially in the case that more EMG signals are needed for acquisition. In Config1, the number of in-amps and filters is equal to the number of EMG signals. In Config2, from 9 to 16 EMGs one more SoC should be employed. In the multiplexing solution of the present invention (Config3), the only requirement is to replace the analog multiplexer with a bigger one. In the case of using a SoC that has integrated AMUX, the present invention can interface more EMG signals without increasing the size of the analog front-end (AFE) at all. It is important to highlight that for the configuration of the present invention (Config3), extra EMG inputs can be added without the use of extra analog to digital converters (ADCs).

In analog front-end systems, power consumption is mainly from the analog to digital converter and the amplifiers. Also, in the overall EMG signal acquisition process, a significant chunk of power consumption stems from the microcontroller unit (MCU).

In Config1, the instrumentation amplifiers (in-amps) and the microcontroller's analog to digital converter (ADC) significantly contribute to the power consumption. The power consumption for each instrumentation amplifiers (in-amp) was 170 uA. Thus, for the 8 in-amps included in this configuration, the power consumption was 1.36 mA. The power consumption value for a SAR analog to digital converter in this configuration was 100 uA, keeping in mind that the analog to digital converter (ADC) is not switched on for the whole data acquisition time period. Under the tests performed by the inventors of the present invention, the analog to digital converter was switched on during 200 us and during this time period the power consumption of the analog to digital converter (ADC) was 100 uA. As a result of the foregoing measurements, the total power consumption of the Config1 analog front-end (AFE) was 1.46 mA.

In the case of Config2, it is not possible to have a separate description of the power consumption by the amplifiers and analog to digital converters. It is only possible to measure the total power consumption in total. Thus, the power consumption in Config2 depends on the selected integrated circuit (SoC) which means that the total power consumption of this analog front-end configuration (AFE) is approximately equal to the power consumption of the integrated circuit (SoC). The lowest power consumption measured for an integrated circuit (SoC) in Config2 was 4.5 mA.

In the multiplexing solution of the present invention (Config3), the total power consumption is from the instrumentation amplifier (in-amp), the microcontroller unit's analog to digital converter (ADC), from the multiplexer switch.

In Config3 of the present invention, only one instrumentation amplifier (in-amp) is used, and the consumption of this amplifier was measured to be about 200 uA. The instrumentation amplifier (in-amp) of the present invention was selected also according other required characteristics, like fast response and high CMRR.

Regarding the power consumption of the analog to digital converter (ADC) in the present invention, a low power SAR can be used. The power consumption of the SAR was measured to be 100 uA. As discussed above with regard to Config1, this calculation was done having in mind that the SAR is not switched on during the whole data acquisition period. In more detail, each one of the eight EMG signals is switched every 25 us which means that for the eight EMG signals, the measured time was 25 us*8=200 us. The sampling took place every 1 ms, which means that for the rest of this period (800 us) the analog to digital converter (ADC) is in sleep mode and is not consuming energy. For the time period that the analog to digital converter (ADC) was switched on, the power consumption by this component was measured to be 100 uA.

Finally, the power consumption of the multiplexer switches in the present invention was measured to be approximately 20 us.

In view of the foregoing measurements, the total power consumption of the analog front-end (AFE) configuration of the present invention (Config3) was 320 uA. Thus, the analog front-end (AFE) configuration of the present invention (Config3) outperforms by far Config1 and Config2 in terms of power consumption.

Table 2 below summarizes the power consumption of the three configurations. With regard to battery life for the three configurations, lower power consumption means longer battery life or the ability to select a smaller battery leading to a further reduction of the device's size which is advantageous for wearable applications.

TABLE 2
AFE Power consumption table (UNK means.

| | Configuration 1 | | Configuration 2 | Configuration 3 |
|---|---|---|---|---|
| Amplifier(s) | 170 uA*8 | | UNK | 200 uA |
| | (=1.36 mA) | | | |
| ADC | 100 | uA | UNK | 100 uA |
| Switches | UNK | | UNK | 20 uA |
| total | 1.46 | mA | 4.5 mA | 320 uA |

In order to estimate the cost of the three analog front-end (AFE) configurations, appropriate components were selected to provide the desired performance. In Config1, standard instrumentation amplifiers (in-amps) and operational amplifiers (op-amps) frequently used in typical analog front-end (AFE) implementations were chosen. In Config2, the cost analysis was based on the ADS1*9* series manufactured by TI.

The lowest cost solution is the analog front-end (AFE) configuration of the present invention (Config3). First, Config1 uses 8 instrumentation amplifiers (in-amps) while the configuration of the present invention (Config3) uses one amplifier which means that it has the ⅛ of the cost of Config1 in terms of amplifier costs.

Config2 uses an expensive chip (SoC) which is not used by the present invention. Also, Config2 includes multiple analog to digital converters (ADCs) whereas in Config1 and the present invention (Config3), the microcontroller's analog to digital converter (ADC) can be used which is standard in most microcontrollers. Thus, there is no extra cost for an external analog to digital converter (ADC) component in the present invention.

The advantages of the present invention (Config3) over the other two configurations (Config1 and Config2) are summarized in Table 3.

TABLE 3
Comparison of the three (3) AFE configurations.

| | Config1 | Config2 | Config3 |
|---|---|---|---|
| Gain | High | Very Low | High |
| Cost | High | Very High | Ultra Low |
| Size | High | Medium | Small |
| Power | Medium | High | Ultra Low |
| consumption | | | |
| Battery life | Medium | Short | Long |
| Extra pins cost in | Medium | High | Very Low |
| adding EMG signals | | | |
| 3^{rd} electrode | Reference/noise | reference | Reference/noise |
| option | cancellation | | cancellation |
| ADC | Flexible/low | Delta-Sigma | Flexible/low |
| characteristics | power ADC | ADC | power ADC |
| Amps number | 8 (or more) | 8 (or more) | 1 |
| ADCs number | 1 | 8 (or more) | 1 |

The means for acquiring EMG signals in the present invention include electrodes that are separately produced from the apparel. Thus, the electrodes of the present invention can be mass produced to fit different types of apparel from different manufacturers. The only variations being the type of apparel (e.g., shorts v. shirts) and the size of the apparel (e.g., small, medium, large etc.). The ability to produce the electrodes in large production quantities, and the simple electrode/fabric fusion process of the present invention reduces the manufacturing cost of apparel with the electrodes integrated thereon.

The electrodes of the present invention utilize a multi-material methodology such that the electrodes of the present invention are stretchable, machine washable, and machine tumble-dryable. The unobtrusive design of the electrodes of the present invention optimize the skin-electrode interface for providing stable and powerful holding onto the skin of the user. Thus, the electrode 8bs of the present invention provide improved fabric adhesion, fabric conformity, and electrode-skin interface. This makes the electrodes of the present invention comfortable when contacting a user's skin, convenient, and durable.

FIG. 8A shows a sensor (6) of the present invention formed by printing multiple layers of conductive-ink and non-conductive insulator ink material. Methods that can be used for printing the multiple layers of ink include, for example, silkscreen, flexography, etc. The multilayered sensor (6) comprises a protective insulation layer (1) of non-conductive insulator ink. The non-conductive insulator ink may be comprised, for example, of a water-based ink or a plastisol-based ink. The protective insulation layer (1) is a substrate that seals the conductive-inks from other elements such as, for example, dirt, skin-cells, water, chemicals, etc. The protective insulation layer (1) is printed onto a heat-transfer film (16) for use as a transport agent for the sensor (6). The heat-transfer film (16) can be made from materials such as, for example, polyester.

A protective conductive layer (2) and a conductive layer (3) are then printed onto the protective insulation layer (1). The conductive layer (2) and conductive layer (3) form a pair of electrodes (6a, 6b) and their wiring. The protective conductive layer (2) of each electrode (6a, 6b) is made from a protective conductive ink such as, for example, flexible silver-based ink or silver-chloride based ink, and the conductive layer (3) of each electrode (6a, 6b) is made from a highly conductive-ink such as, for example, silver-based conductive ink. The conductive layer (3) acts as a biometric electrode capable of capturing biometric signals. The conductive layer (3) exhibits great interface with the skin of a user.

The multilayered sensor (6) further comprises a protective insulation layer (4) which may include some adhesion functionality. The protective insulation layer (4) is comprised of materials such as, for example, a water-based ink or plastisol-based ink. The multilayered sensor (6) also comprises an adhesion layer (5) made from materials such as, for example, water-based adhesive or plastisol-based adhesive. These additional prints of non-conductive insulator ink are applied over the conductive-inks to seal the sensory material from other elements such as, for example, dirt, skin-cells, water, chemicals, etc. and to provide adhesion between the apparel and the sensor (6).

A multilayered interface (7) which merges several sensor leads (15) together is shown in Figure. The multilayered interface (7) comprises a protective insulation layer (1), a protective conductive layer (2), a conductive layer (3), a protective insulation layer (4) which may include some adhesion functionality, and an adhesion layer (5).

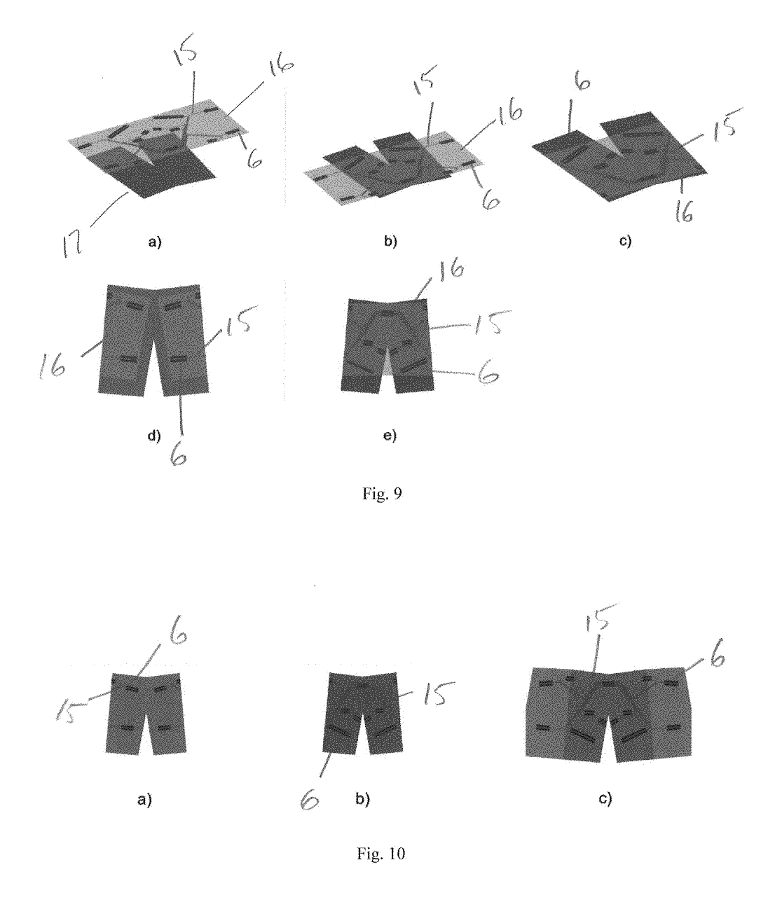

The heat-transfer film (16) to which the sensors (6) are printed onto is heat-pressed onto the apparel (17) for permanently bonding the printed sensors (6) to the fabric of the apparel (17). As shown in FIG. 9A, the apparel (17) is turned inside-out and the heat-transfer film (16) is aligned to the apparel (17) so that the electrodes (6a, 6b) of the sensors (6) are located at positions corresponding to muscles of interests when the apparel (17) is being worn by a user. The heat-transfer film (16) is then placed atop the apparel (17) as shown in FIG. 9B. The heat-transfer film (16) is wrapped around the apparel (17) as shown in FIGS. 9C, 9D, and 9E, respectively, and then heat-pressed for a short period of time. After the heat-press process is completed and the heat-transfer film cools down, and the heat-transfer film (16) is removed leaving the printed sensors (6) permanently bonded to the apparel as shown in FIGS. 10A, 10B, and 10C.

In order to be worn comfortably, wearable devices which calculate, monitor, and send the bio-signal information obtained from the sensors to separate display devices such as a smartphones should be small and easy to wear. In addition, the size of the connector/socket which merges the signal leads together for connection with the wearable device should be small. Although some wearable devices have reduced size, bulky connectors substantially increase their final dimensions. The added bulkiness resulting from the connector limits the possible placement options on apparel because there are few places on the body where a big connector can be placed without causing problems of mobility for the user. Another limitation found in most connectors is the small number of inputs which means that even if the hardware is able to acquire many signals, the end connector would be too large for anyone to wear on an item of apparel.

The connector/socket (12) of the present invention is durable and has a minimal footprint which means that it can be placed on many locations around an article of apparel. Hence, the connector (12) can be located near many different places of the human body when the apparel is being worn. Exemplary dimensions of the reduced size of the connector/socket (12) are a width of about 6 cm, a length of about 5.6 cm, and a height of about 0.65 cm. The connector (12) of the present invention also increases the number of signals that can be sent to a central device (13), maintains signal quality, reduces the cost and time for manufacturing, and simplifies the sensor integration on apparel so that no special machinery is required.

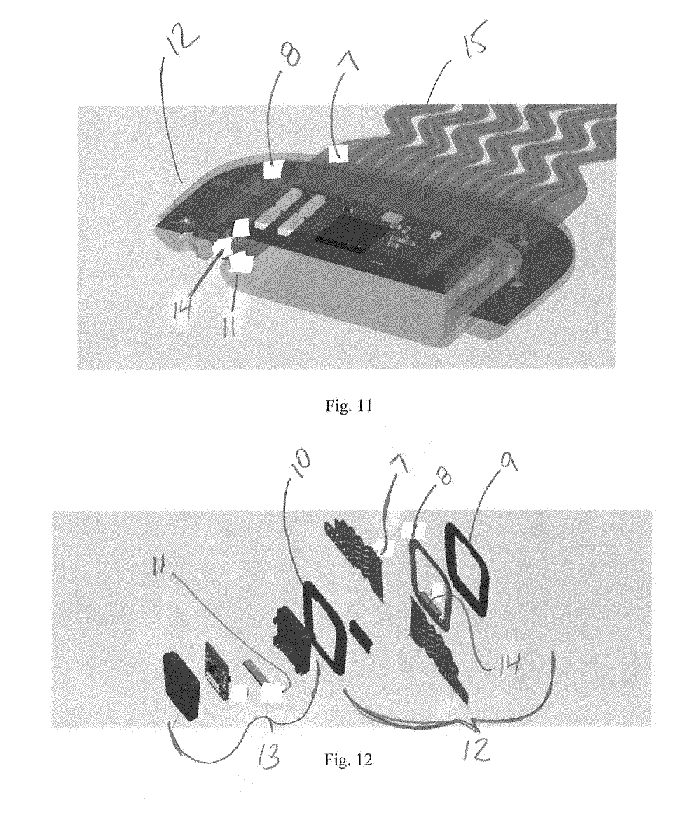

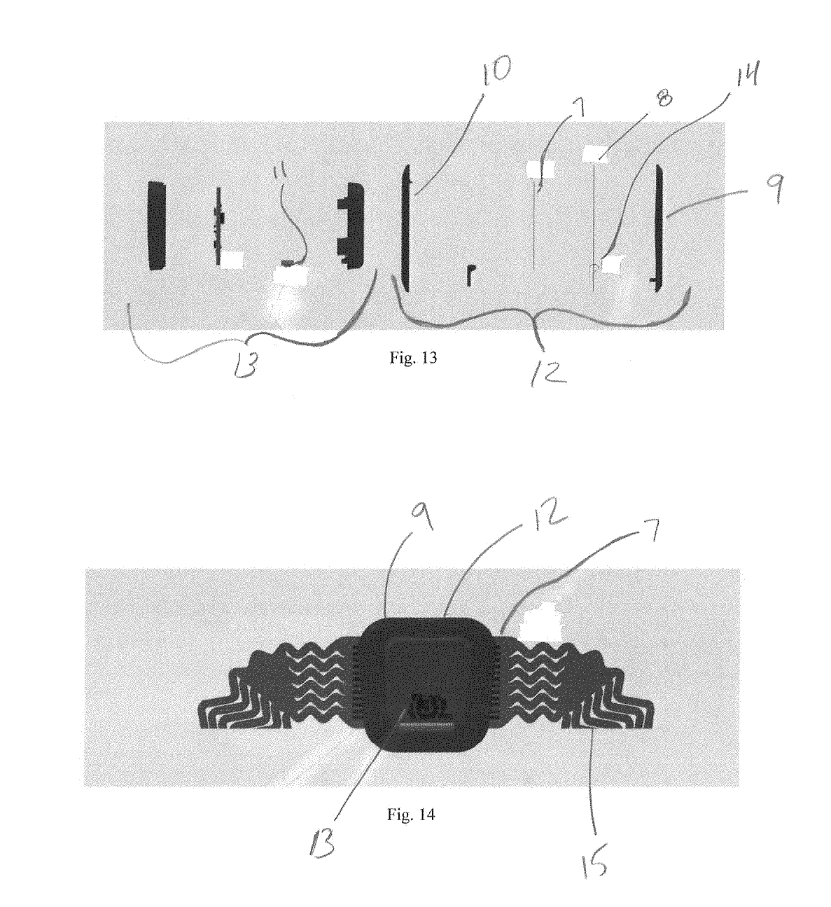

As shown in FIGS. 11-14, the connector (12) comprises an interface (7) which combines sensor leads (15) from each of the sensors (6) together. The interface (7) is firmly secured atop a printed circuit board (PCB) (8) of the connector (12). The interface (7) and the printed circuit board (8) are covered by interior and exterior coverings (9, 10) to secure placement, signal-integrity, and water-resistance. Signals from the sensors (6) are sent through the interface (7) to a lead (14) of the printed circuit board (PCB) (8). The lead (14) is firmly secured in the connector (12) and is in constant contact with the elastomeric connector (11) of the central device (13) when the central device (13) is plugged into the connector (12). This completes the connector (12), central device (13) interface.

In this way, the connector (12) sends the signals from the sensors (6) directly to the central device (13) with the minimum amount of steps possible while achieving minimal size and cost. It also achieves an improved user experience as the connector (12) is of a minimal size when the central device (13) is not being worn, and when the central device (13) is being worn, the connector (12) and the central device (13) are still of a manageable size so as to not hinder the user in any way when exercising.

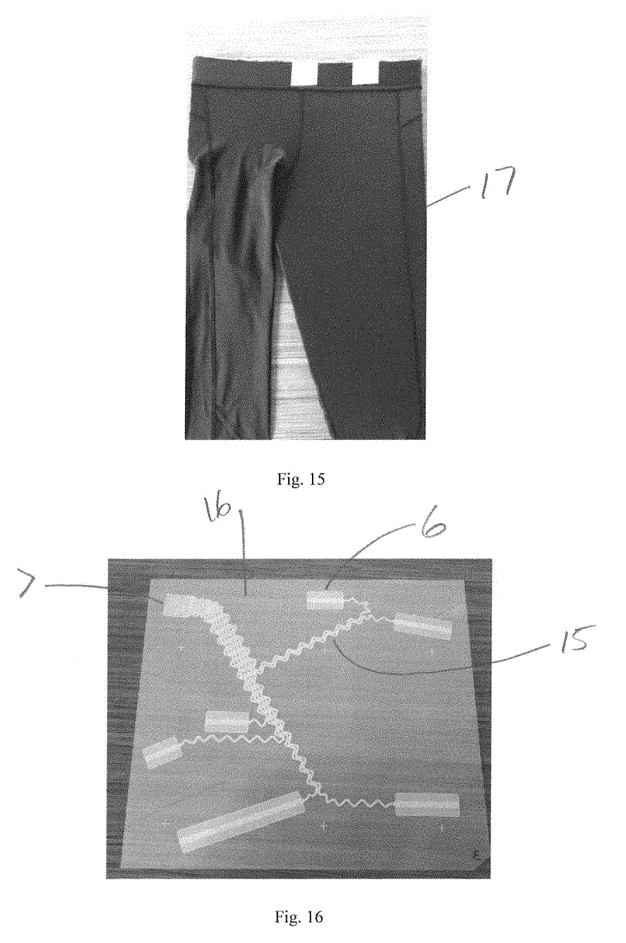

FIGS. 15-23 show the steps of the sensor integration on an item of apparel (17). These figures are exemplary in nature and are not intended to limit the scope of the present invention. For example, the article of apparel (17) shown in FIGS. 15-23 is a pair of exercise pants, and it is contemplated that different types of apparel such as, for example, upper garments may also include the sensor integration of the present invention for monitoring muscle activity for different parts of the human body.

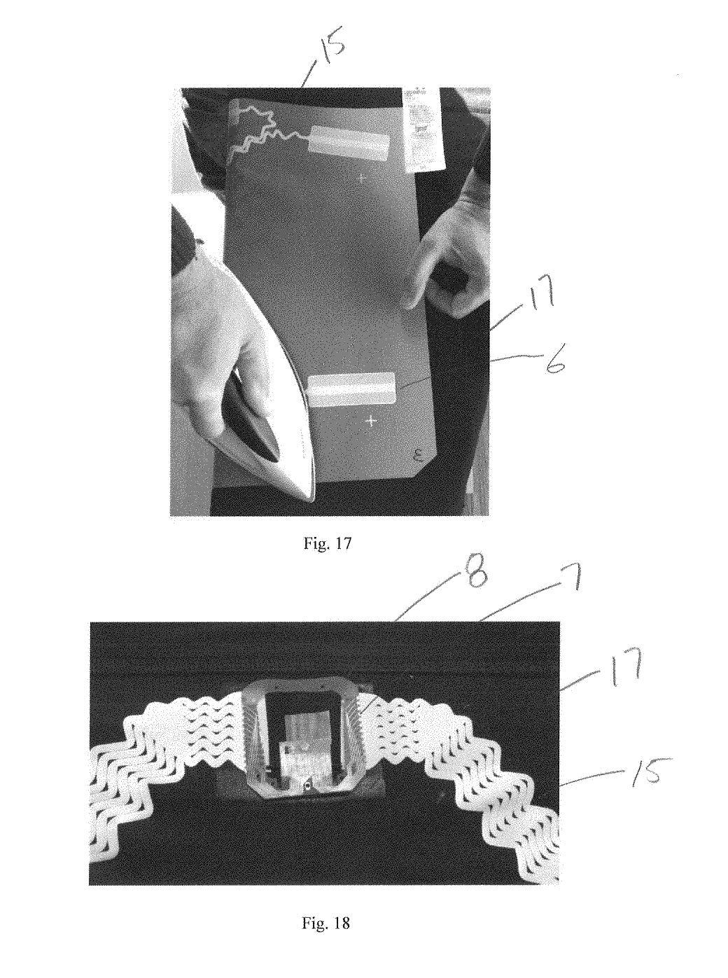

FIG. 15 shows an item of apparel (17) before sensor integration, i.e. before the sensors (6) and sensor leads (15) are attached to the apparel (17). FIG. 16 shows the heat-transfer film (16) with the sensors (6) and sensor leads (15) printed thereon. FIG. 17 shows heat-pressing the heat-transfer film (16) to permanently bond the sensors (6) and the sensor leads (15) from the heat-transfer film (16) to the piece of apparel (17). After the sensors (6) and sensor leads (15) are permanently bonded to the apparel (17), the heat-transfer film (16) is removed. FIG. 17 shows heat-pressing the heat-transfer film (16) by way of a hand iron typically used for ironing clothing. This is an example of just one way to heat-press the sensors (6) and sensor leads (15) onto an article of apparel (17), and additional methods for heat-pressing the sensors (6) and sensor leads (15) onto apparel are possible, especially industrial pressing methods.

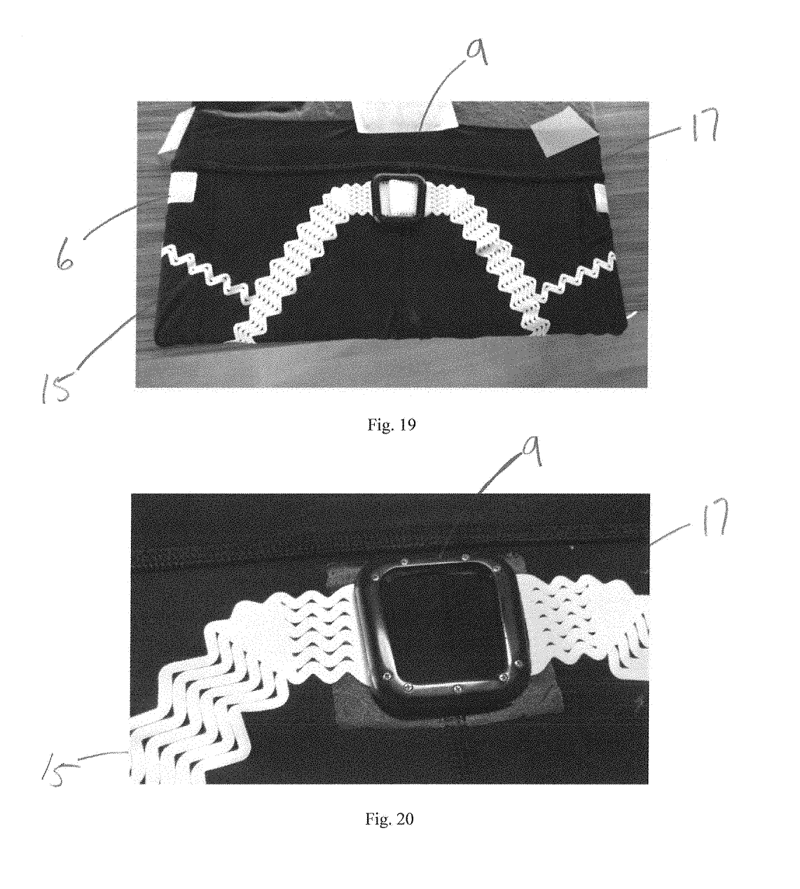

FIG. 18 shows the placing of a printed circuit board (PCB) (8) onto the interface connector (7) which merges the sensor leads (15) integrated on the article of apparel (17) together. FIGS. 19 and 20 show an interior view of the article of apparel (17) with an interior cover (9) secured over the printed circuit board (PCB) (8) and interface connector (7). FIGS. 19 and 20 also show the sensors (6) and sensor leads (15) permanently bonded onto the interior of the article of apparel (17).

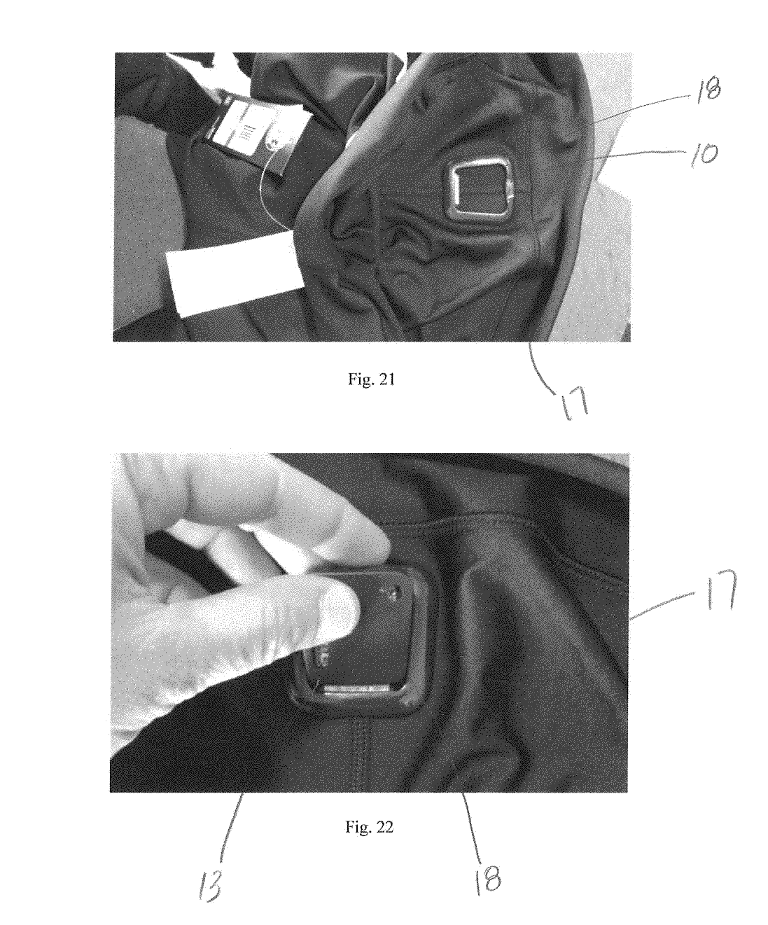

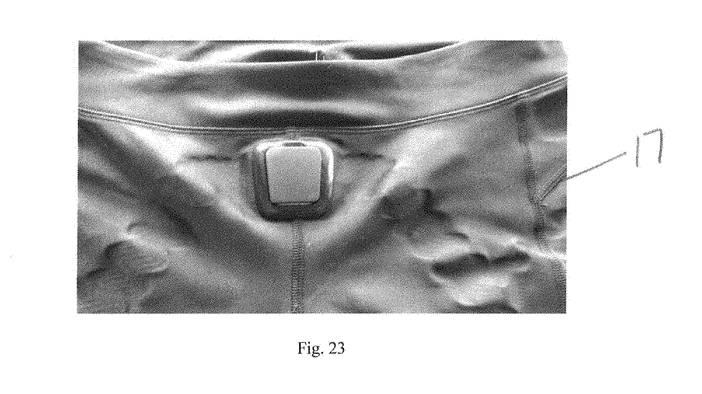

FIG. 21 shows an exterior image of the connector (12) with an exterior cover (10) on the piece of apparel (17). Finally, FIG. 22 shows a view of a central device (13) being plugged into the connector (12). When the central device (13) is successfully secured into the connector (12), a connection between the central device (13) and the sensors (6) is created. The central device (13) comprises the analog front-end system, and a processor and memory for collecting, computing, and forwarding measurements obtained from the sensors (6). The central device (13) is configured to forward data wirelessly such as, for example, via Bluetooth technology so that the data can be sent to a display screen for viewing by the user. As an example, the data can be sent to a smartphone for viewing by the user. Thus, the central device (13) uses the analog front-end system to collect the EMG signals obtained from the sensors (6) integrated onto the apparel (17), and is configured to send this data to a display screen for viewing by the user. For example, the data can be sent to a mobile device with a display screen for viewing by the user.

It is further contemplated that the central device (13) can also track and monitor the movement of the user. Also, in addition to monitoring the electrical signals generated by the muscles of the user, the sensors (6) can also obtain data on additional body signals such as heart rate, respiratory rate, body temperature etc. Thus, in addition calculating biometrics relating to muscle output, the central device (13) can also calculate, monitor, and forward data on activity level, heart rate, breathing rate, calories burned, and steps taken by a user wearing the apparel (17) with sensors (6) integrated thereon, and central device (13) plugged into connector (12).

While various embodiments of the present invention have been described above, it should be understood that they have been presented by way of example, and not limitation. It will be apparent to persons skilled in the relevant art(s) that various changes in form and detail can be made therein without departing from the spirit and scope of the present invention. Thus, the present invention should not be limited by any of the above-described exemplary embodiments, but should be defined only in accordance with the following claims and their equivalents.

What is claimed is:

1. An analog front-end system for the acquisition of a plurality of EMG signals for assessing the health of muscles and nerve cells that control the muscles, comprising:
(a) means for acquiring the plurality of EMG signals and delivering a differential signal for each of the plurality of EMG signals;
(b) a first set of filters comprising a first filter for each of the plurality of EMG signals;
(c) a multiplexer;
(d) a single amplifier; and
(e) a microcontroller unit comprising an analog-to-digital converter;
wherein the bandwidth of each of the plurality of EMG signals is filtered by the first set of filters before the EMG signals are multiplexed by the multiplexer, and
wherein the EMG signals are multiplexed by the multiplexer so that the single amplifier can be used for amplifying the EMG signals before they reach the analog-to-digital converter to provide a gain of at least 250 times, and wherein the signal can be retrieved by the analog-to-digital converter with a quantization error of less than 0.012%.

2. The analog front-end system of claim 1, wherein
the analog front-end system further comprises a second set of filters comprising a second filter for each of the plurality of EMG signals.

3. The analog front-end system of claim 2, wherein
the first set of filters are passive anti-aliasing filters and the second set of filters are passive high pass filters.

4. The analog front-end system of claim 1, wherein
the amplifier is a low power instrumentation-amplifier with high Common Mode Rejection Ratio.

5. The analog front-end system of claim 1, wherein
the first set of filters comprise eight first filters and the second set of filters comprise eight second filters for filtering eight EMG signals.

6. The analog front-end system of claim 2, wherein
the EMG signals pass through the first set of filters and the second set of filters to restrict the bandwidth of the EMG signals to a frequency range of 20 Hz to 100 Hz before the EMG signals are multiplexed by the multiplexer.

7. The analog front-end system of claim 7, wherein
the multiplexer is an analog switch and the operation of the multiplexer is controlled by the microcontroller.

8. The analog front-end system of claim 1, wherein
the multiplexer, the amplifier, and the microcontroller unit are part of an integrated circuit.

9. The analog front-end system of claim 1, wherein
the system is implemented into the fabric of sports apparel for long term monitoring of EMG signals during physical activity.

10. The analog front-end system of claim 1, wherein the means for acquiring the plurality of EMG signals and delivering the differential signal for each of the plurality of EMG signals comprise multilayered signals, wherein said multilayered signals comprise:
a first protective insulation layer of non-conductive insulator ink;
a protective conductive layer and a conductive layer forming a pair of electrodes;
a second protective insulation layer; and
an adhesion layer.

11. The analog front-end system of claim 10, wherein
the conductive layer of said multilayered signals is made from a highly conductive-ink for capturing biometric signals.

12. The analog front-end system of claim 10, wherein
the protective insulation layer of said multilayered signals is printed onto a heat-transfer film for use as a transport agent for said multilayered signals.

13. The analog front-end system of claim 10, wherein
said multilayered signals permanently bond to a fabric when the heat-transfer film to which said multilayered signals are printed onto is heat-pressed onto the fabric.

14. A method for processing a plurality of EMG signals for assessing the health of muscles and nerve cells that control the muscles comprising the steps of:
(a) acquiring a differential signal for each of the plurality of EMG signals,
(b) applying a first set of filters comprising a first filter for each of the plurality of EMG signals to restrict the bandwidth of each of the plurality of EMG signals to a predetermined frequency range,
(c) multiplexing the filtered EMG signals by an analog multiplexer switch,
(d) amplifying the multiplexed EMG signals by a single differential amplifier, and
(e) sending the amplified EMG signals to a microcontroller unit for processing,
wherein the bandwidth of each of the plurality of EMG signals is filtered by the first set of filters before the EMG signals are multiplexed by the multiplexer, and
wherein a gain of at least 250 times and a quantization error of less than 0.012% is obtained before the EMG signals are retrieved by the analog-to-digital converter.

15. The method according to claim 14, further comprising the step of
applying a second set of filters comprising a second filter for each of the plurality of EMG signals before multiplexing the EMG signals.

16. The method according to claim 15, wherein
the first and second filters restrict the bandwidth of each of the plurality of EMG signals to a frequency range of 20 Hz to 100 Hz.

17. The method according to claim 14, wherein
eight differential EMG signals are processed.

18. The method according to claim 14, wherein
the differential amplifier is a low power instrumentation-amplifier with high Common Mode Rejection Ratio.

19. The method according to claim 15, wherein
the first set of filters are passive anti-aliasing filters and the second set of filters are passive high pass filters.

20. The method according to claim 14, wherein
the processing comprises an analog to digital conversion by an analog-to-digital converter.
